(19) United States
(12) Patent Application Publication
(10) Pub. No.: US 2022/0409134 A1
(43) Pub. Date: Dec. 29, 2022

(54) Using an In-Ear Microphone Within an Earphone as a Fitness and Health Tracker

(71) Applicant: Google LLC, Mountain View, CA (US)

(72) Inventors: Ant Oztaskent, London (GB); Yaroslav Volovich, Cambridge (GB)

(21) Appl. No.: 17/342,092

(22) Filed: Jun. 8, 2021

(51) Int. Cl. A61B 5/00; H04R 1/10; H04R 1/40; H04R 3/00; H04R 1/08; G10L 25/66; A61B 5/11; A61B 7/00; A61B 5/08; G16H 50/20

(52) U.S. Cl. CPC A61B 5/6817; H04R 1/1016; H04R 1/406; H04R 3/005; H04R 1/08; G10L 25/66; A61B 5/7267; A61B 5/1118; A61B 7/00; A61B 5/0823; A61B 5/4205; A61B 5/0816; G16H 50/20; A61B 2562/0219; A61B 2562/0204; A61B 2560/0257

(57) ABSTRACT

Trained machine learning models can be used for analysis of signals obtained through an in-ear or on-body device. Signals can be analyzed to determine information related to activities such as eating, chewing, drinking, coughing, or sneezing. In addition, data from an in-ear thermometer or other data sensors can be analyzed in conjunction with the machine learning models to provide data or recommendations to a user on a user device or initiate an action.

### BACKGROUND

Health and fitness tracking modalities and devices have become more commonplace. These modalities can track certain physical parameters of a user, such as blood pressure, blood oxygen level, or heart rate. In addition, in-ear devices, such as earbuds or earpods have also become ubiquitous. These devices may contain a speaker for allowing a user to listen to media, a bluetooth connection to allow for communication with another device, and a microphone to allow for external audio signals to be picked up.

### SUMMARY

Aspects of the disclosed technology include methods, systems, and apparatuses for using an earphone as a fitness tracker and health tracker.

Aspects of the present disclosure Aspects of the disclosed technology can include an electronic device, the electronic device comprising one or more sensors, one or more processors; and one or more non transitory computer readable media. The one or more non-transitory computer-readable media can store instructions that when executed by the one or more processors cause the electronic device to perform operations. The operating can comprise receiving sensor data generated by at least one sensor of the one or more sensors that is at least partially positioned within an ear of a user, wherein the sensor data was generated by the at least one sensor concurrently with a voluntary user activity and processing the sensor data with a trained machine learning model to generate an interpretation of the voluntary user activity as an output of the trained machine learning model. The at least one sensor comprises one or more microphones that convert a sound wave located within an ear canal of the ear of the user to the sensor data. The sound wave located within the ear canal of the ear of the user is generated by an eardrum of the user. When the one or more microphones are placed within the ear of the user, the one or more microphones can be directed toward the eardrum of the user. The at least one sensor comprises one or more of an accelerometer; a gyroscope; a RADAR device; a SONAR device; a LASER microphone; an infrared sensor; or a barometer. The electronic device can be sized and shaped to be at least partially positioned within the ear of the user. The electronic device can be coupled to an ancillary support device, such as, for example, a user device, a cell phone, a smart phone, or other electronic device that is physically separate from at least one sensor. The interpretation of the sensor data as an output by the trained machine learning model can categorize the sensor data as one or more of coughing, breathing, chewing, sneezing, swallowing, or drinking. The interpretation of the output by the trained machine learning model can comprise a classification of the output into one or more of a plurality of categories. The plurality of categories comprise a plurality of defined user states. The operations can comprise determining, by analysis of additional health data obtained from a health sensor, whether an emergency condition related to the user is met.

Aspects of the disclosed technology can include an ear bud, earphone, or other wearable device, which can include at least one sensor, one or more processors; and one or more non-transitory computer-readable media that store instructions. The instructions, when executed by the one or more processors, can cause the ear bud to perform operations. The operations can comprise receiving sensor data generated by the at least one sensor, wherein the sensor data was generated by the at least one sensor concurrently with user activity detectable through signals associated with a portion of the user's anatomy proximate the ear and processing the sensor data with a machine-learned model to generate an interpretation of the sensor data as an output of the machine-learned interpretation model. The at least one sensor can comprise a microphone can convert a sound wave located within an ear canal of the ear of the user to the sensor data. The sound wave can be located within the ear canal of the ear of the user and can be generated by an eardrum of the user. The microphone can be placed within the ear of the user, the microphone is directed to the eardrum of the user. The interpretation of the sensor data can be an output by the trained machine learning model and can categorize or analyze the sensor data as coughing, breathing, chewing, sneezing, swallowing, or drinking. The interpretation of the output by the machine-learned interpretation model can comprise a classification of the output into one or more of a plurality of categories. The plurality of categories can comprise a plurality of defined user states. The operations can comprise determining, by analysis of additional health data obtained from a health sensor, whether an emergency condition related to the user is met.

Aspects of the disclosed technology can comprise a method that can comprise receiving sensor data generated by the at least one sensor, wherein the sensor data was generated by the at least one sensor concurrently with user activity detectable through signals associated with a portion of the user's anatomy proximate the ear and processing the sensor data with a machine-learned model to generate an interpretation of the sensor data as an output of the machine-learned interpretation model. The sensor data is based on sound waves produced by vibrations within the ear of the user resulting from the user's action. The sensor can be part of an in-ear device. The in-ear device can be coupled with at least one ancillary support device. The trained machine learning model can be implemented in the in-ear device, in at least one ancillary device, or in both devices. In some examples, a “first stage” and a “second stage” or “first step” and “second step” models can be used in one or both devices. In some examples, the “first stage” or “first step” model or filter can be executed in the in-ear device or other electronic device in real-time or near real time, while the “second step” model, which in some examples may be more computationally intensive, and can be executed in real-time, in near-real time, or non-real time periods, or during periods of downtime or processor availability of the ancillary device. The second model can provide an output to a user. The trained machine learning model, the first stage model, or second stage model can be implemented in at least one ancillary support device.

Aspects of the disclosed technology can include a system that can at least one sensor configured to be at least partially positioned within an ear of a user and to generate sensor data concurrently with user activity detectable through signals associated with a portion of the user's anatomy proximate the ear, one or more processors; and one or more non-transitory computer-readable media that store instructions that when executed by the one or more processors cause the one or more processors to perform operations. The operations can comprise receiving the sensor data generated by the at least one sensor and processing the sensor data with a trained machine learning model to generate an interpretation of the user activity as an output of the trained machine learning model. Health data obtained from a health sensor can be processed in conjunction with the sensor data. The sensor can be part of an in-ear device of the system. The in-ear device can be coupled with at least one user device of the system. Any part of one or more trained machine learning models, the trained machine learning model, or sub-models can be implemented in the in-ear device or another connected device.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1A depicts a graphical diagram of an example device, such as an in-ear device, positionable within or near an ear of a user according to aspects of the present disclosure.

FIG. 1B depict block diagrams of example computing systems that include an in-ear device according to aspects of the present disclosure.

FIG. 2A is an illustration of a wearable user device capable of health functions according to aspects of this disclosure.

FIG. 2B is a diagram of user interfaces according to aspects of this disclosure.

FIG. 2C is a schematic diagram of communication between devices according to aspects of this disclosure.

FIG. 3 depicts an example architecture for training one or more machine-learning models based on sound signals.

FIG. 4 depicts a block diagram of an example computing system for training machine learned models according to examples of the present disclosure.

FIG. 5 depicts an example method according to aspects of the present disclosure.

Reference numerals that are repeated across plural figures are intended to identify the same features in various examples and figures.

### DETAILED DESCRIPTION

### Overview

The disclosed technology in one aspect may comprise detection tools which utilize aspects of machine learning and artificial intelligence to allow an earbud or other wearable device to act as a fitness tracker or health tracker. In some examples, the earbud may be an “in-ear” earbud. The earbud may contain a microphone or other sensor generally configured to obtain signals or sounds external to the user such as sounds or other signals captured through aerial conduction. A second microphone or other sensor can be included in the earbud and configured to detect sounds from within a user's ear canal, such as those present during chewing, coughing, or swallowing. The earbud may also contain a thermometer which is internally facing, allowing for continuous and more confident monitoring of a user's body temperature from a consistent location (e.g. the inner ear canal).

In some examples, the disclosed technology may be used to derive health related information, such as water or, more generally, liquid consumption, food consumption, breathing related information (e.g. breathing rate, breathing depth, breathing regularity), sneezing (such as allergic sneezing, virus induced sneezing, and bacteria induced sneezing), and body temperature information. In some examples, the detected health related information can be obtained through the use of trained machine learning models.

In some examples, such as through analysis of a signal (e.g. sound or air pressure), the opening and closing of an eustachian tube can be detected and related to certain actions, such as swallowing, chewing, yawning, or sneezing. In some examples, certain signatures or classifications associated with an action can be used to classify a signal detected through an earbud. For example, the opening and closing of the eustachian tube can be used to identify a particular type of action. This information can be included or used in training a machine learning model.

In some examples, models or algorithms using multi-stage detection can be used. For example, in some examples, a first stage event such as a sneezing, swallowing, chewing, or coughing can be detected using a first model and a second model can be used to interpret the event with more detail, such as to track illness, allergies, choking, or to potentially initiate an emergency response. In some examples, the second model can be chosen based on the type of first stage event and/or the presence or availability of additional health sensors or health sensor data.

In additional examples, additional algorithms or machine learning models can be used to combine the derived health related information with other health sensors or other data. Non-limiting examples of information which may be combined by obtaining from other health sensors or deriving data from other health sensors include gyroscope data, oxygen in oxyhemoglobin and deoxyhemoglobin, heart arrhythmias, heart rate, premature ventricular contractions, missed beats, systolic and diastolic peaks, and large artery stiffness index, sleep data, electrodermal activity (EDA) activity. The combination of additional data can identify periods of activity which may affect or interact with the information derived from the earbud information.

As a non-limiting example, signals collected via the earbud can be used to estimate an amount or type of liquid consumed by a user. For example, a trained machine learning model can be used based on information provided by the user to a mobile application which records the amount and type of liquid consumed. The trained machine learning model can take as inputs the type of liquid consumed, available health sensor data, and signals obtained from the earbud to train a machine learning model. The trained machine learning model can then be used to classify or detect the amount of liquid consumed.

As another example, a trained machine learning model can be used to detect a quantity of food being consumed or the constituent components of the food being consumed. For example, a trained machine learning model can be used which can detect the time over which food was being consumed, the quantity of food consumed, or the relative proportions of carbohydrates, proteins, and fats contained in the food consumed.

As another example, a user's breathing pattern can be derived from signals obtained by the in-ear earbud to estimate an activity level of the user. The deepness or shallowness of breathing, the speed of breathing, and regularity or irregularity of breathing can be used to estimate an activity level. This information can be used in conjunction with other information obtained through other health sensors, such as heart rate, blood pressure, or gyroscope information to identify the level of activity, calorie burn, or whether variations in the breathing is caused by allergens, a medical event (e.g. pain, heart attack, stroke, etc.).

As another example, emergency responses can be initiated based on the output of a first or second machine learning model.

An aspect of the technology is the training and updating of models associated with processing the inputs. In some examples, techniques which anonymize or keep user information local to the user device (e.g. stored securely locally without sharing to the cloud or a server) but allow for the additional data generated by multiple users to be used in updating or training models can be used. As one example, Federated Learning (FL) approaches can be used. For instance, FL enables mobile devices to collaboratively learn a shared prediction model while maintaining the training data locally on the device. As such, in accordance with the disclosed technology, a mobile device may download the shared model from, for example, the cloud. The shared model may then be updated by learning from events that take place on the mobile or other user device. In effect, the original shared model may become more personalized to the user. An anonymized and locally trained model, without underlying user data, on user devices may be saved as updates that are then sent back to the cloud to update the shared model. The updated shared model may then be provided back to individual user devices. As the use of FL ensures that all the training data remains on each user device, user privacy is ensured.

Although examples given within are with respect to devices such as in-ear devices, other devices which are capable of obtaining similar signals by being positioned proximate to or on the user's skin, neck, jawline, face, nose, etc. can also be used as aspects of the disclosed technology. As one example, a device sitting near the skull, jaw, or other area can be used to detect signals which are conducted via bone. As another example, over-ear devices with an internal microphone can be used to detect vibrations from the inner ear or vibrations transmitted through the skull of a user. Similarly, other signals, such as other vibrations produced during voluntary activities, such as from the jaw (e.g. jaw joint vibrations) can be used as additional signals for analysis by trained machine learning models.

As used in this disclosure voluntary user activities can include activities over which a user has a degree of control, including but not limited to, sneezing, eating, chewing, coughing, breathing, and swallowing.

### Example Systems

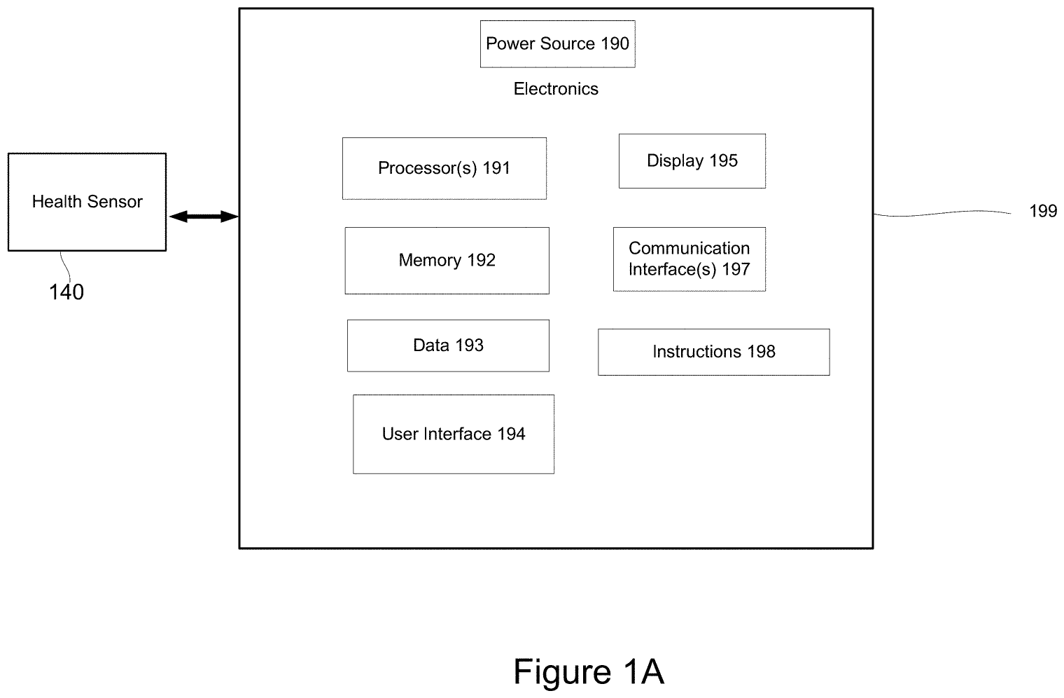

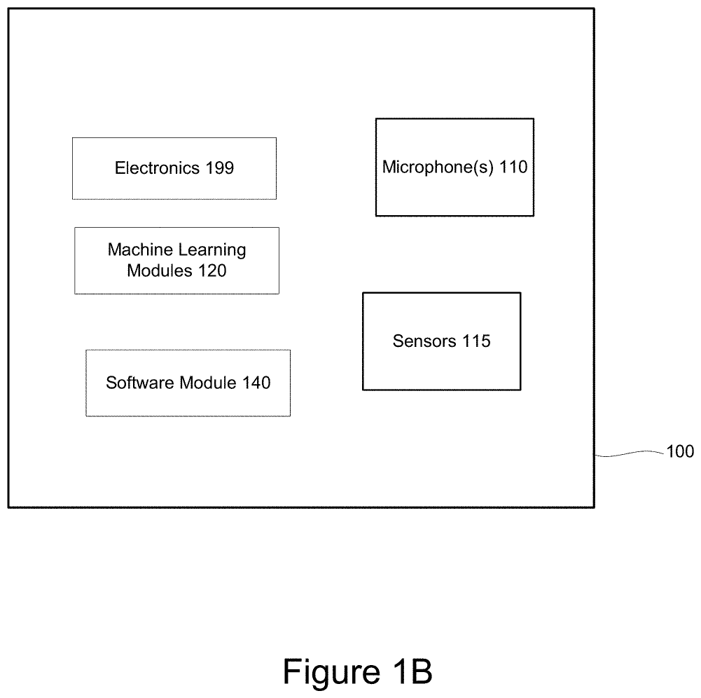

FIG. 1 illustrates additional aspects of electronics 199, which may be used in aspects of the disclosed technology as described in further detail below. Electronics 199 can be any computing device which is capable of performing the steps and algorithms described herein, such as without limitations, cell-phones, tablets, computers, laptops, servers, smart devices, or smart watches. Although the description in FIG. 1 is given with respect to electronics 199, a person of skill in the art should understand that in some examples electronics 199 can be combined or operate collectively with health sensor 140. Illustrated in FIG. 1 is a bidirectional arrow indicating that communication(s) between a health sensor 140 and electronics 199 can occur.

Health sensor 140 can be any device, circuitry, or module which can be used to observe or determine information related to a health state of a user, such as, for example, blood pressure, blood oxygen levels, stress, or other metrics which can be derived from a combination of the exemplary aforementioned metrics. Health sensor equipment, such as an analog front end, photodetectors, accelerometers, or health sensors, such as photoplethysmography sensors, devices, or circuitry. In some examples, a health sensor need not be part of the same device as electronics 199, and can be included in a separate device. It is to be understood that although health sensor 140 is illustrated with a specific configuration, other arrangements of these components are within the scope of this disclosure. In other examples, health sensor 140 can be included or arranged within user devices, such as a mechanical watch, a smart watch, a smart ring, a cell phone, earbud, headphone, armband, or a laptop computer. In other examples, health sensor 140 can be integrated into jewelry, such as a pendant, necklace, bangle, earring, armband, ring, anklet, or other jewelry.

Electronics 199 may contain a power source 190, processor(s) 191, memory 192, data 193, a user interface 194, a display 195, communication interface(s) 197, and instructions 498. The power source may be any suitable power source to generate electricity, such as a battery, a chemical cell, a capacitor, a solar panel, or an inductive charger. Processor(s) 191 may be any conventional processors, such as commercially available microprocessors or application-specific integrated circuits (ASICs); memory, which may store information that is accessible by the processors including instructions that may be executed by the processors, and data. Memory 192 may be of a type of memory operative to store information accessible by the processors, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The subject matter disclosed herein may include different combinations of the foregoing, whereby different portions of the instructions and data are stored on different types of media. Data 193 of electronics 199 may be retrieved, stored or modified by the processors in accordance with the instructions 198. For instance, although the present disclosure is not limited by a particular data structure, data 193 may be stored in computer registers, in a relational database as a table having a plurality of different fields and records, XML documents, or flat files. Data 193 may also be formatted in a computer-readable format such as, but not limited to, binary values, ASCII or Unicode. Moreover, data 193 may comprise information sufficient to identify the relevant information, such as numbers, descriptive text, proprietary codes, pointers, references to data stored in other memories (including other network locations) or information that is used by a function to calculate the relevant data.

Instructions 198 may control various components and functions of health sensor 140. For example, instructions 198 may be executed to selectively activate light source 110 or process information obtained by photodetector 120. In some examples, algorithms can be included as a subset of or otherwise as part of instructions 198 included in electronics 199.

Instructions 198 can include algorithms to interpret or process information received from the health sensor 140 or from other parts of electronics 199, such as information received through or generated by analyzing health information from health sensor 140, information in data 193, information displayed on display 195, or information processed by processors 191. For example, physical parameters of the user can be extracted or analyzed through algorithms. Without limitation the algorithms could use any or all information about the waveform, such as shape, frequency, or period of a wave, Fourier analysis of the signal, harmonic analysis, pulse width, pulse area, peak to peak interval, pulse interval, intensity or amount of light received by a photodetector, wavelength shift, or derivatives of the signal generated or received by a photodetector of health sensor 140. Other algorithms can be included to calculate absorption of oxygen in oxyhemoglobin and deoxyhemoglobin, heart arrhythmias, heart rate, premature ventricular contractions, missed beats, systolic and diastolic peaks, and large artery stiffness index. In yet other examples, artificial learning or machine learning algorithms can be used in both deterministic and non-deterministic ways to extract information related to a physical condition of a user such as blood pressure and stress levels, from, for example, heart rate variability. PPG can also be used to measure blood pressure by computing the pulse wave velocity between two points on the skin separated by a certain distance. Pulse wave velocity is proportional to blood pressure and that relationship can be used to calculate the blood pressure. In some examples, the algorithms can be modified or use information input by a user into memory of electronics 199 such as the user's weight, height, age, cholesterol, genetic information, body fat percentage, or other physical parameters. In other examples, machine learning algorithms can be used to detect and monitor for known or undetected health conditions, such as an arrhythmia, based on information generated by the photodetectors, health sensors, and/or processors.

Instructions 198 can also include trained machine learning modules which can be used to determine whether a sensor is present or included on a user device, to detect information related to voluntary user actions (e.g. chewing, eating, speaking, swallowing, drinking), breathing patterns, “first-stage” detections (e.g. classifying or categorizing a signal as relating to a particular activity, such as eating, coughing, speaking, swallowing) and “second-stage” detections (e.g. combining multiple signals or inputs from different sensors, detecting the amount of food consumed, amount of liquid consumed, the depth of breathing, the number of times swallowed, how rapidly a user is chewing).

User interface 194 may be a screen which allows a user to interact with health sensor 140, such as a touch screen or buttons. Display 195 can be an LCD, LED, mobile phone display, electronic ink, or other display to display information about health sensor 140. User interface 194 can allow for both input from a user and output to a user. In some examples, the user interface 194 can be part of electronics 199 or health sensor 140, while in other examples, the user interface can be considered part of a user device. User interface 194 may also comprise devices such as keyboards, etc.

Communication interface(s) 197 can include hardware and software to enable communication of data over standards such as Wi-Fi, Bluetooth, infrared, radio-wave, and/or other analog and digital communication standards. Communication interface(s) 197 allow for electronics 199 to be updated and information generated by health sensor 140 to be shared to other devices. In some examples, communication interface(s) 197 can send historical information stored in memory 192 to another user device for display, storage, or further analysis. In other examples, communication interface(s) 197 can send the signal generated by the photodetector to another user device in real-time or afterwards for display on that device. In other examples, communication interface(s) 197 can communicate to another PPG module. Communication interface(s) 197 can include bluetooth, Wi-Fi, Gazelle, ANT, LTE, WCDMA, or other wireless protocols and hardware which enable communication between two devices.

FIG. 1B illustrates a graphical diagram of an example device 100. In some examples, device 100 can be an in-ear device, such as an earbud or in-ear microphone. Device 100 can be positioned at least in part in an outer ear and/or at least in part in the inner ear such as an ear canal. Device 100 can thus be partially contained within an ear canal or a user. Device 199 can contain any combinations of components described with respect to electronics 199 (FIG. 1A). As illustrated in FIG. 1B, electronics 199 are illustrated as a block.

Device 100 can include one or more sensors, as described herein with reference to FIG. 1B, which can generate or capture sensor data that describes phenomena occurring related to the user or the user's ear, such as within a user's ear canal. As examples, the phenomena can include movement, vibrations, audio signals, visual changes, tissue deformations, and/or other phenomena within the ear, such as within the ear canal.

As one example, illustrated in FIG. 1B, device 100 can also contain other sensors, such as, for example, a microphone 110 which is configured to be directed towards the ear canal or ear drum of a user. Microphone 110 may be positioned within the ear 20, for example at least partially within the ear canal. Microphone 110 of device 100 can record audio signals occurring within the ear canal that are caused by the user performing actions, such as coughing, breathing, chewing, eating, or swallowing. For example, the audio signals can be at least in part generated by vibration of an eardrum, bone conductions, and/or other vibrating structures/tissues. In some examples, microphone 110 can be configured to take advantage of the opening and/or closing of the eustachian tube which occurs when a user performs certain actions, such as chewing, swallowing, or eating. In some examples, distinct signatures can be obtained through this opening and/or closing based on particular movements. In some examples, this can form the basis of an additional or supplemental trained machine learning model, the output of which can be used in conjunction with the output of other models.

Device 100 can include one or more sensors 115. At least some of the sensors 115 can generate sensor data that describes phenomena occurring in the ear of the user. As one example, the sensors can include one or more microphones that convert a sound wave located within an ear canal of the ear of the user to the sensor data. For example, when the device 100 is placed within the ear of the user, the one or more microphones can be directed toward the eardrum of the user. In some examples, device 100 can include one or more additional microphones that are not located within the ear canal. For example, the additional microphone(s) can collect audio signals that are external to the user's ear, including speech of the user from the user's mouth.

In some examples, the additional microphone(s) can be used to filter out audio signals from a source external to the user from the audio signals recorded by the one or more microphones directed toward the user. Depending on the volume and frequency of signals from sources external to the user, these signals may leak into the signals recorded by the one or more microphones directed to the user. To compensate for these additional signals, the additional microphone(s) can record external audio that can be later identified and removed from the signals recorded by the one or more microphones directed toward the user.

The accuracy of the one or more microphones in converting a sound wave can be improved in some cases by the shape, size, or intended position of device 100 when worn by the user. In some examples, the in-ear device 100 is shaped so that when worn as intended, device 100 sits flush in the ear canal of the user. In these examples, the shape of device 100 can help to focus the microphone(s) in picking up sound waves within the ear canal of the user instead of sound waves from sources external to the user.

As further examples, in addition or alternatively to the microphone(s), the sensors 115 can include some or all of the following: an accelerometer; a gyroscope; a RADAR device; a SONAR device; a LASER microphone; an infrared sensor; and/or a barometer. For example, these sensors can detect and record motion/acceleration, such as by an accelerometer; a gyroscope; physical deformations in the ear such as deformations/vibrations of the eardrum, such as by a RADAR device, a SONAR device, a LASER microphone, or an infrared sensor; and/or pressure changes in the ear, such as by a barometer. The sensor data collected by any or all of these sensors 115 can be used in addition or alternatively to the use of audio data captured by one or more microphones as described herein.

As yet further examples, the one or more sensors 115 can include a thermometer and/or a proximity sensor, such as a sensor that features a conductive coating. Device 100 can analyze the data generated by these sensors to determine whether or not the device 100 is currently placed within the user's ear. If the device is not within the user's ear, certain features such as collection of sensor data can be paused until such time as device 100 is placed into the ear.

In some examples, the device 100 can use, store, or implement trained machine learning models or machine learned models, which can be included on the device or be stored and implemented on a related or connected device. Machine-learned models can be or can otherwise include various machine-learned models such as neural networks or other types of machine-learned models, including non-linear models and/or linear models. Neural networks can include feed-forward neural networks, recurrent neural networks such as long short-term memory recurrent neural networks, convolutional neural networks or other forms of neural networks. Example machine-learned models 120 are discussed further infra.

In some examples, device 100 can store and implement a software module 140. In some examples, software module 140 can be an AI-based personal assistant which can perform certain operations or take certain actions on behalf of the user, for example, based on input received from the user. One example of an AI-based personal assistant is the Google Assistant. In other examples, software module 140 can be or contain speech to text recognition capabilities to take actions on behalf of a user's speech commands. In other examples, the software module can cause another application such as a phone call application, text message, email application, music application, or other applications which can be run in conjunction with another computing device. In some examples, software module 140 can cause the applications to perform various operations, for example through one or more application programming interfaces.

Device 100 can also include one or more speakers. Speakers can be formed through the operation of magnets and drivers to form sound waves. The speakers can be used to convey audio information to the user, such as when music is played, phone calls are being made, or prompts are being returned to the user.

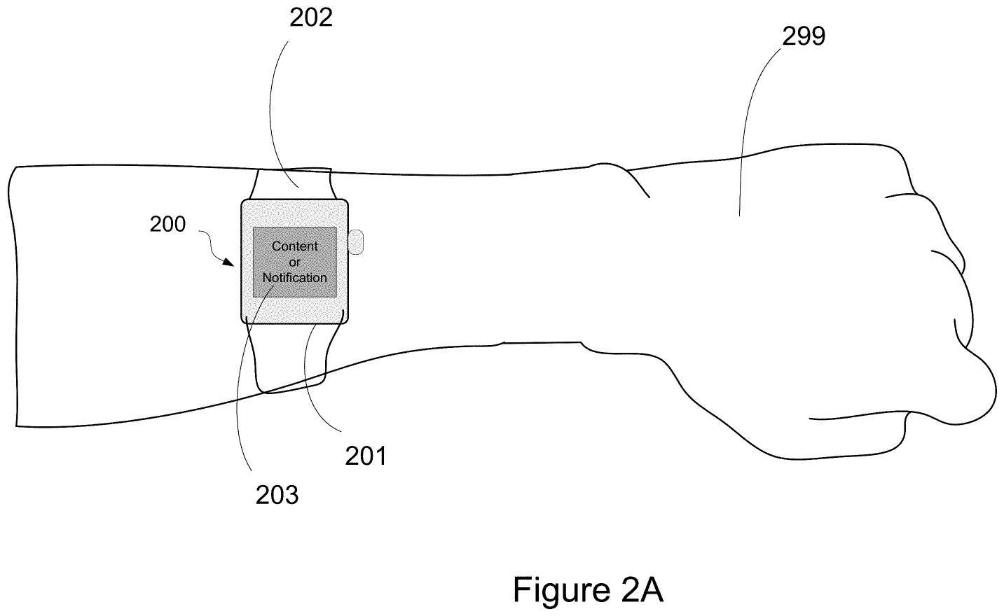

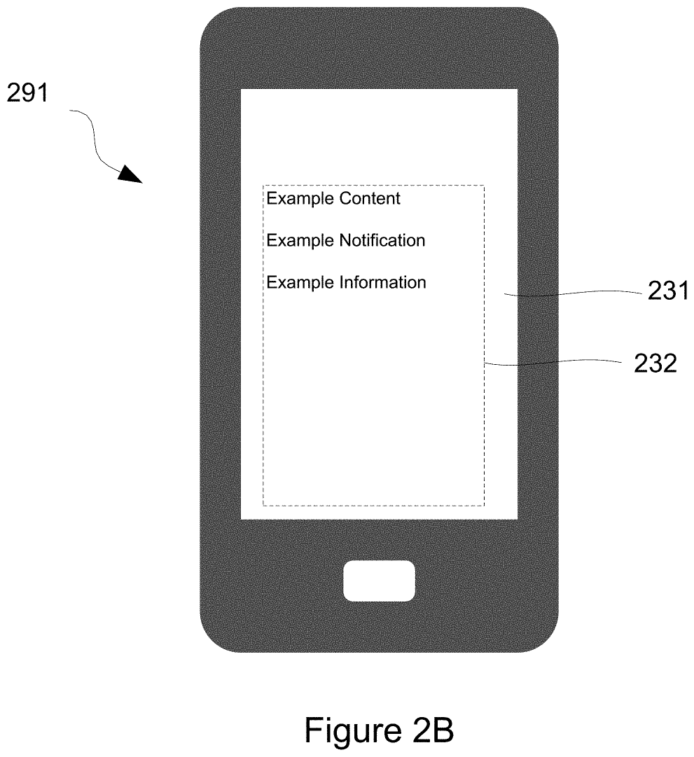

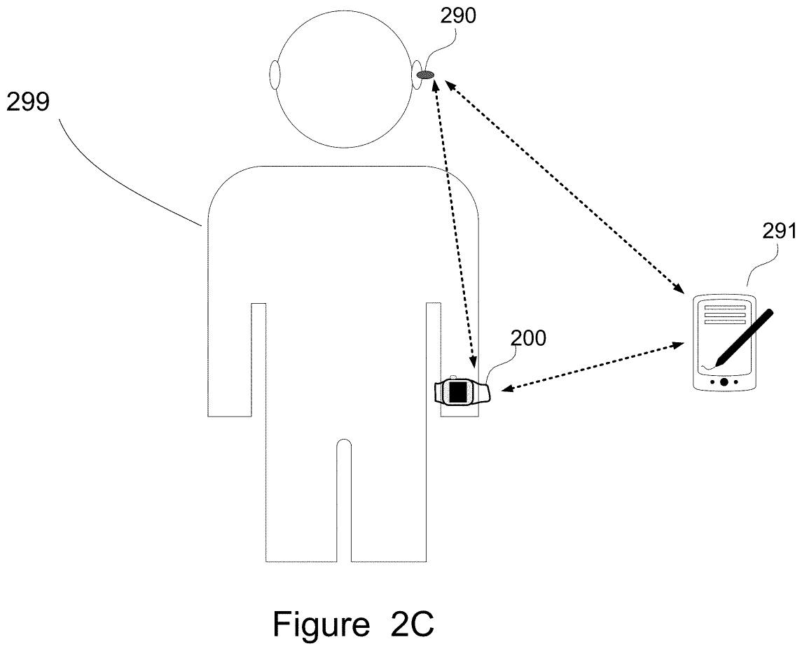

FIG. 2A illustrates a user device, 200, which can be worn by a user, such as user 299. The user device can include a housing 201, and a strap 202. Housing 201 can have components such as a back portion, which will contact the skin of user 299. The back portion can contain a glass portion which will allow light to pass through the back portion. For example, light can be generated from other components contained within housing 201, such as a light source. User device 200 and housing 201 can also have a user interface which allows user 299 to interact and view information from user device 200. The user interface can be part of a touchscreen or other device. Additional components which can be included in user device 200 or in housing 201 are further described above with reference to FIG. 1. The housing can further be of an appropriate thickness to include the components described in FIG. 1. Strap 202 can be a strap to hold the user device on a user, such as one made from metal, leather, cloth, or other material. User device 200 can contain a health sensor module 140 to perform health sensing functions.

Although a smartwatch is illustrated as user device 200, a person of skill in the art will appreciate that user device 200 can take on a variety of forms. User device 200 can also be a health sensor, an earbud or earplug, headphone, or other wearable electronics, a ring, a bangle, an anklet, necklace, or other piece of jewelry. A notification on user device 200 can be a visual notification, such as on display 203 of the user device while in other examples, according to the capabilities of the user device, other notifications can be given such as through a vibration, an audio alert, a beep, a flash, or other notification.

FIG. 2B illustrates a user device 291. User device 291 can contain various components described with reference to FIG. 1 in electronics 199, which are omitted from FIG. 2B for simplicity. Illustrated in FIG. 2B is display 231 displaying content 232. Content 232 can include textual content, visual data, such as images or videos, as well as meta-data which may or may not be displayed on user device 230.

In some examples, user device 291 can be used to record information which can be used to train a machine learning algorithm or machine learning model. For example, a user may track his or her food consumption (e.g. when the user eats, the estimated composition of food consumption, or amount of food eaten) or amount of liquid consumption. In other examples, a user may provide user specific information, such as age, gender, body weight, eating habits, activity levels, which can all be used to fine-tune or further modify a machine learning model.

FIG. 2C illustrates communication between multiple user devices, user device 200, user device 291 (e.g. a computing device including smartphone, tablet, laptop, server computing device, computing device that can be worn, embedded computing device, vehicle computing device, navigational computing device, gaming console), and user device 290 worn by user 299. In one example, user device 290 is an earbud. Although the same reference numerals are used for the devices in FIG. 2C as the devices in FIGS. 2A-2B, these devices need not be the same devices. In other examples, user device 290 can be similar to device 100. As explained further below, each user device can compute or derive user related or health related information, and a combination of the information can be used when making determinations related to the user. For example one device may determine heart rate while another determines blood pressure or blood oxygen level, while user device 100 determines if a person is breathing heavier or irregularly. In some examples, a device not containing a health sensor, such as smartphone 291 can receive health related information from user device 200 and user device 290 and combine information related to the two devices and analyze or run algorithms, including but not limited to machine learning algorithms, to generate information related to user 299. In some examples, raw signals, partially processed data, or digitized data can be obtained by one user device, but analyzed or processed by another user device. For example, raw or processed sound data from a microphone of user device 290 and temperature data from a thermometer of user device 290 can be combined with heart rate data obtained from user device 200 and be processed by user device 291.

In some examples, some or all of the components described with reference to user device 200 or user device 100 can be located in another user device (e.g. user device 200 or user device 291).

### Example Methods

As explained below, the following techniques or methods can be used to analyze data obtained from sensors, health devices, or other user devices, applications, or inputs to, without limitation, detect, quantify, or determine user activity.

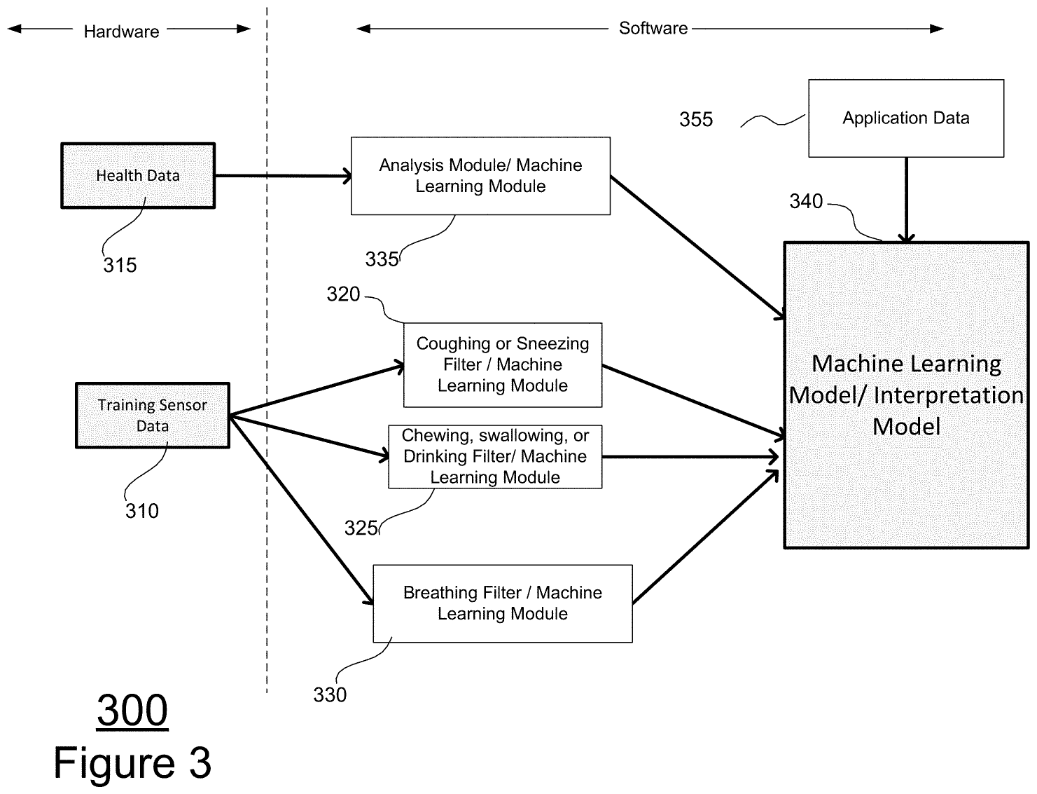

FIG. 3 illustrates an example schematic view of architecture 300. FIG. 3 illustrates how training sensor data can be used to train a machine learning model. Although a specific architecture and example implementation is provided with reference to FIG. 3 to train a machine learning model, other techniques, approaches, and architectures can be used to train a machine learning model.

Illustrated in FIG. 3 is training sensor data 310. Training sensor data 310 can include data which has been obtained from a user device, such as user device 100 or user device 290. Training sensor data can include without limitation sound signals or sound data, audible signal or sound data, gyroscope signals or data, motion signals or data, temperature signals or data, or any other data which can be obtained from or generated from user device 100 or user device 290. Training sensor data can be filtered or categorized before being provided to the machine learning model for training, such as through a coughing or sneezing filter 320, a chewing or drinking filter 325, or a breathing filter 330. These filters can classify whether the sound or data being provided belongs to one of these specific categories. In some examples, these filters can operate by analyzing a part or whole of data in training sensor data 310. The aforementioned filters operate to classify or provide additional information regarding the information contained in training sensor data 310 and in some examples, additional filters or no filters can be used. In some examples, the filters can use time information to classify the training sensor data by relating a particular signal with a particular time.

In some examples the filters can be machine learning models themselves. The filters can act as a “first step” model which can identify a type of action being performed by the user while the “second step” model can be a model configured for more precision or act as a “finer” model to identify details or other information within the action. For example, a first step model or filter can identify that a user is “drinking” while the second step can identify the type of drinking being consumed, such as a fizzy or effervescent drink or non-fizzy drink. As another example, a first step model can identify “chewing” while the second step model can identify the speed or rate of chewing.

In some examples, the aforementioned filters can default to a signal being a “breathing signal” or breathing related data if a coughing, sneezing, chewing, or drinking classification or determination is not made.

In other examples, techniques can be used to convert or modify the training sensor data 310 into different formats. For example, machine learning model 340 can convert audio data into spectrograms. A spectrogram is a visual representation of an audio signal over some domain. For example, the spectrogram can represent changes in frequency of the audio signal visually over a period of time. The machine learning model 340 can process the spectrogram to generate additional features from the training sensor data. For example, the machine learning model 340 can generate features from spectrograms representing changes in amplitudes caused by variations in frequency in the training sensor data 310 as indicated in the spectrograms.

One reason for converting sensor data 204 into different formats is to be able to leverage feature extraction techniques particular to a given format. For example, by converting incoming sensor data 204 to spectrograms, the model 202 can leverage any of a variety of different machine learning feature generation techniques for image data, which may not have been applicable to the incoming sensor data 204.

Health data 315 can be obtained from, for example, health sensor 140 or user device 200. Health related statistics, features or parameters, e.g., heart rate data, heart rate variability data, sleep data, PPG data, etc., are also provided to the model. Such data may be provided from a wearable device as signals or may be derived from signals provided by the wearable device. In some examples, module 335 can include additional specialized information for analysis depending on the type of health data 315 available. Health data 315 can be provided to an analysis module 335 for analysis, formatting, or combination or derivation of other health related metrics.

Application data 355 can include information provided by a user, such as for example, the information provided with reference to FIG. 2B. This information can include the amount and type of liquid consumed, food portions, food types, or constituents of food consumed, weight, height, gender, and age. In some examples, this information can be used for determining a ground truth interpretation for training sensor data related to the application data, such as, data training sensor data generated at the same time or near in time to the application data being provided.

Machine learning model 340 can be trained using the various inputs described above. In some examples, machine learning models trained elsewhere can be an unsupervised learning model. In some examples, a “ground truth” can be established when using training data to train a machine learning model. In some examples, the “ground truth” can be provided by a user when the user is performing an activity. For example, while data is being collected, a user can indicate which type of activity the user is performing, such as eating, drinking, or chewing via a user interface or application. In other examples, a separate trained machine learning model can be used to produce or determine ground truth data. As one example, a machine vision model used in conjunction with a camera can be used to determine or categorize an activity that a user is engaging in, such as chewing or swallowing. The ground truth can thus be established by labeling the data through the machine learning model. In other examples, manually annotated or known data can be used as part of a collection within a laboratory or other controlled environment to establish ground truth data.

In some examples, multiple models can be trained within machine learning model 340. For example, a “two-stage” model can be used, in which a first model can be trained to make a “general” classification of the type of event being indicated by the data (e.g. sneezing, breathing, chewing), and a second model trained based on the event (e.g. detecting the cause of the sneezing or level of congestion, the breathing pattern or rate, or type of food being eaten). In these examples, the training or use of multi-tiered models can be used to advantageously perform a quick classification of a signal, and perform a more computationally intensive analysis. In other examples, machine learning model 340 can be a “second stage” model while another model can act as a filter or “first stage” model as described herein.

In some examples, machine learning model 340 can be trained using a supervised learning approach. Training sensor data 310 can include sets of sensor data, such as audio signals captured by microphones, gyroscope data, temperature data, or other data, which were collected while a person was performing an action or generating that data with a known ground truth interpretation, which can be included in the data. An interpretation can be obtained from machine learning model 310 on each set of training sensor data 310. The interpretation can be compared to a ground truth interpretation for the data which is known. The machine learning model can then further be trained or adjusted using a loss function to compute a gradient of a loss function with respect to parameter values of machine learning model 340, such as for example, the weights of a neural network.

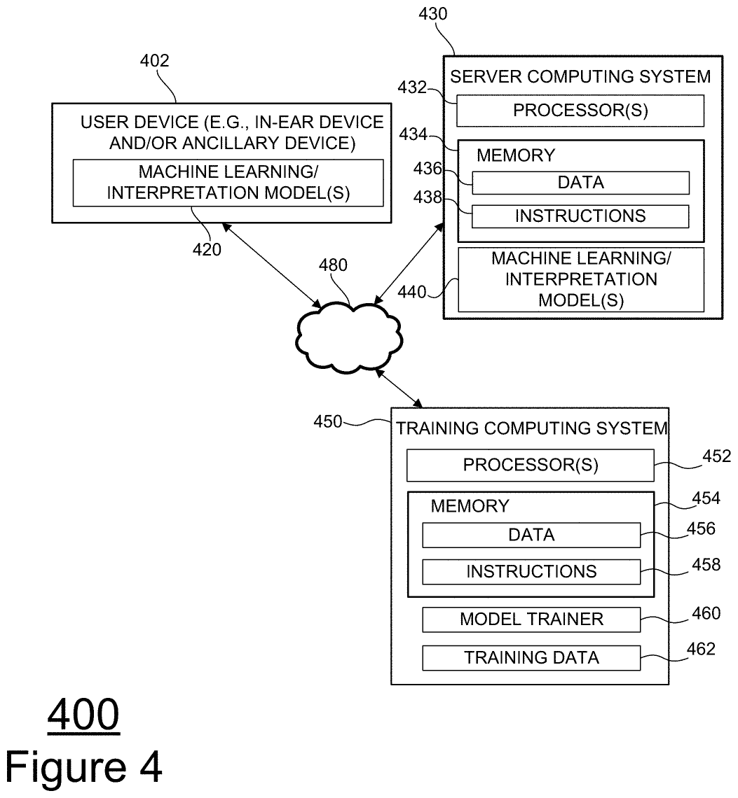

FIG. 4 depicts a block diagram of an example computing system 400 for training machine-learned models according to example examples of the present disclosure. The system 400 includes a user computing device 402, a server computing system 430, and a training computing system 450 that are communicatively coupled over a network 480. As described with reference to FIG. 4, the trained interpretation models can be similar to the trained machine learning model described with reference to FIG. 3, and vice versa.

The user computing device 402 can be any type of computing device, such as, for example, a personal computing device, such as a laptop or desktop computer, a mobile computing device, such as a smartphone or tablet, a gaming console or controller, a wearable computing device, an embedded computing device, or any other type of computing device.

In some examples, the user computing device 402 can store or include one or more trained or untrained machine learning models 420, such as those described herein. For example, the trained or untrained machine learning models 420 can be or can otherwise include various machine-learned models such as neural networks, such as deep neural networks, or other types of machine-learned models, including non-linear models and/or linear models. Neural networks can include feed-forward neural networks, recurrent neural networks, such as long short-term memory recurrent neural networks, convolutional neural networks or other forms of neural networks. Other examples of machine learning models or machine learning techniques that are included herein can be included in computing device 402.

In some examples, the one or more trained or untrained machine learning models 420 or interpretation models 420 can be received from the server computing system 430 over network 480. These models can be stored and implemented at the user computing device 402. In some examples, the user computing device 402 can implement multiple parallel instances of a single machine-learned model 420, such as to perform parallel audio data interpretation across multiple instances of sensor data, including for example, live sensor data and historic sensor data.

Additionally or alternatively, one or more machine-learned models 440 can be included in or otherwise stored and implemented by the server computing system 430 that communicates with the user computing device 402 according to a client-server relationship. For example, the machine-learned models 440 can be implemented by the server computing system 440 as a portion of a web service, such as an interpretation service. Thus, one or more models 420 can be stored and implemented at the user computing device 402 and/or one or more models 440 can be stored and implemented at the server computing system 430.

The server computing system 430 includes one or more processors 432 and a memory 434. The one or more processors 432 can be any suitable processing device, such as a processor core, a microprocessor, an ASIC, a FPGA, a controller, or a microcontroller, can be one processor or a plurality of processors that are operatively connected. The memory 434 can include one or more non-transitory computer-readable storage mediums, such as RAM, ROM, EEPROM, EPROM, flash memory devices, or magnetic disks, and combinations thereof. The memory 434 can store data 436 and instructions 438 which are executed by the processor 432 to cause the server computing system 430 to perform operations.

In some examples, the server computing system 430 includes or is otherwise implemented by one or more server computing devices. In instances in which the server computing system 430 includes plural server computing devices, such server computing devices can operate according to sequential computing architectures, parallel computing architectures, or some combination thereof.

As described above, the server computing system 430 can store or otherwise include one or more machine-learned models 440. For example, the models 440 can be or can otherwise include various machine-learned models. Example machine-learned models include neural networks or other multi-layer non-linear models. Example neural networks include feed forward neural networks, deep neural networks, recurrent neural networks, and convolutional neural networks.

In some examples, training can occur on a training computer system. The user computing device 402 and/or the server computing system 430 can train the models 420 and/or 440 via interaction with the training computing system 450 that is communicatively coupled over the network 480. The training computing system 450 can be separate from the server computing system 430 or can be a portion of the server computing system 430, such as one or more hardware devices, virtual nodes, or virtual computers. The training computing system 450 includes one or more processors 452 and a memory 454, whether in hardware or virtually. The one or more processors 452 can be any suitable processing device, such as a processor core, a microprocessor, an ASIC, a FPGA, a controller, or a microcontroller and can be one processor or a plurality of processors that are operatively connected. The memory 454 can include one or more non-transitory computer-readable storage mediums, such as RAM, ROM, EEPROM, EPROM, flash memory devices, or magnetic disks, and combinations thereof. The memory 454 can store data 456 and instructions 458 which are executed by the processor 452 to cause the training computing system 450 to perform operations. In some examples, the training computing system 450 includes or is otherwise implemented by one or more server computing devices.

The training computing system 450 can include a model trainer 460 that trains the trained or untrained machine learning models 420 and/or 440 stored at the user computing device 402 and/or the server computing system 430 using various training or learning techniques, such as, for example, backwards propagation or forward propagation of errors. For example, a loss function can be back propagated through the model(s) to update one or more parameters of the model(s), for example based on a gradient of the loss function. Loss functions which can be used can include custom defined loss functions or loss functions which are a combination or mathematical techniques such as mean squared error, cross entropy loss, non-linear loss functions, or other functions. In some examples, gradient descent techniques can be used to iteratively update the parameters over a number of training iterations.

In some examples, performing backpropagation can include performing truncated backpropagation through time. The model trainer 460 can perform a number of generalization techniques, such as decaying weights, or performing dropout, to improve the generalization capability of the models being trained. In particular, the model trainer 460 can train the trained or untrained machine learning models 420 and/or 440 based on a set of training data 462. The training data 462 can include, for example, supervised training data, including user-specific supervised training data generated, sensor data, data provided by the user such as data input through a user device (e.g. smartphone) or data obtained through simulated or generated datasets, such as for example, during a calibration routine.

Thus, in some examples, if the user has provided consent, the training examples can be provided by the user computing device 402. Thus, in such examples, the model 420 provided to the user computing device 402 can be trained by the training computing system 450 on user-specific data received from the user computing device 402. In some instances, this process can be referred to as personalizing the model.

The model trainer 460 includes computer logic utilized to provide desired functionality and can enable the training of untrained machine learning models. The model trainer 460 can be implemented in hardware, firmware, and/or software controlling a general purpose processor. For example, in some examples, the model trainer 460 includes program files stored on a storage device, loaded into a memory and executed by one or more processors. In other examples, the model trainer 460 includes one or more sets of computer-executable instructions that are stored in a tangible computer-readable storage medium such as RAM hard disk or optical or magnetic media. The model trainer 460 can train the interpretation models 420 according to different hyperparameters associated with model training, such as different learning rates, batch sizes, and other hyperparameters associated with the particular type of model implemented, or the particular technique used to train the models 420. The model trainer 460 can also implement any variety of techniques for hyperparameter search or optimization, for example random search, or population- or evolutionary-based techniques on an ensemble of models. The network 480 can be any type of communications network, such as a local area network, such as intranet, wide area network, such as the Internet, or some combination thereof and can include any number of wired or wireless links.

FIG. 4 illustrates one example computing system that can be used to implement the present disclosure. Other computing systems can be used as well. For example, in some examples, the user computing device 402 can include the model trainer 460 and the training dataset 462. In such examples, the models 420 can be both trained and used locally at the user computing device 402. In some of such examples, the user computing device 402 can implement the model trainer 460 to personalize the models 420 based on user-specific data. In other words, the model trainer 460 can train the models 420 offline, online, or according to a hybrid approach.

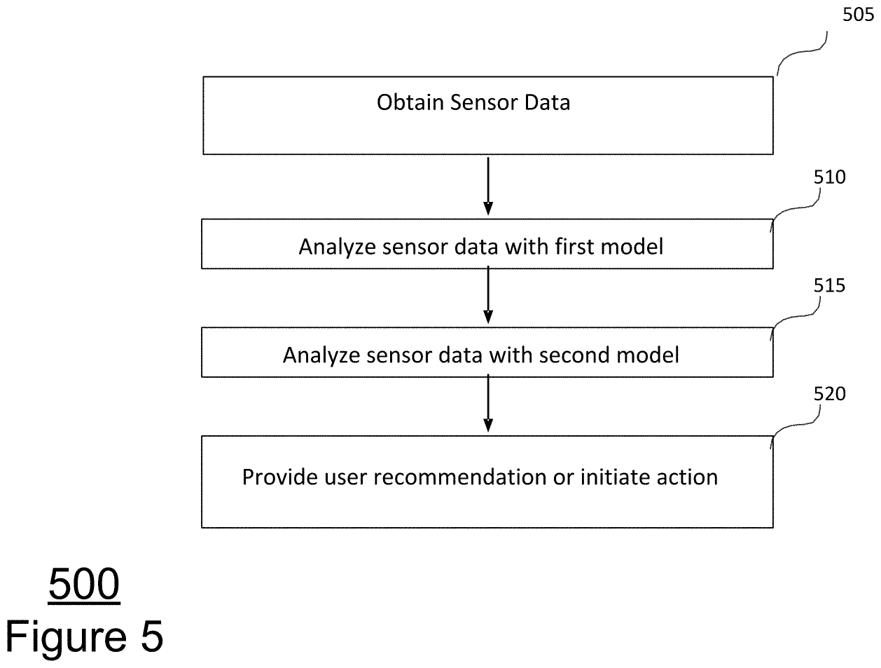

FIG. 5 illustrates a flowchart of an example method 500.

At block 505, sensor data related to a user can be obtained. User data obtained at block 505 can include data described above or similar to training sensor data 310 and health data 315. This data can be obtained or captured using any of the sensors described herein.

At block 510, the obtained sensor data can be analyzed with a first model. In some examples, the first model can be a “lighter” or computationally less intensive model which can analyze the data in real time. For example, the first model can provide a classification of the current state of the user or of the sensor data based on signatures or sampling of the sensor data. For instance, the first model can be a trained machine learning model which can classify the sensor data into a particular category, such as “chewing,” “speaking,” “yawning” or “breathing.” In some examples, the model can default to “breathing” or another default condition of the user if no other signature for another category is detected. In some examples, the first model can be run in real time as long as sensor data is being obtained.

At block 515, sensor data can be analyzed with a second model. In some examples, the second model can be a trained machine learning model. In some examples, the second model can be selected based on the output or classification of the first model. For example, if the first model suspects that the user is drinking, the second model selected can be a “drinking” model which can estimate the amount of liquid consumed. Similarly, if the second model is an eating model, an “eating” model can be selected and used to estimate the amount of food consumed or its type based on the signal data obtained. The second model can be stopped when the first model changes its classification. In some examples, the second model can also incorporate or use information provided from available health sensors, such as health sensor 140. For example, additional information related to heart rate, blood oxygen level, or EDA information can be incorporated in the analysis of the second model.

At block 520, a user recommendation or action can be initiated based on the output of the first or second model. For example, information output from the second model can be sent to a user device where it can be stored or displayed to a user. In other examples, a number of estimated calories burned throughout the day (from a combination of audio information related to breathing and PPG, heart rate, or EDA data) can be stored on a user device. In other examples, if a “choking” condition is detected by the first model, an emergency response can be initiated whereby emergency services are contacted from a user device notifying them of the user's condition. In other examples, if a proximity sensor determines that an earbud or wearable is inserted into the ear canal, but breathing is no longer detected while HR information is still available, an emergency response can be initiated based on the possibility of a stroke or other medical event occurring.

As examples, a user may wake up and use a device, such as an earbud, headphones, or other wearable. The device may be able to automatically detect that the user has worn or inserted the device through a proximity sensor. The user may already be wearing another wearable device, such as a health tracker on his or her wrist. Information from this health tracker may be used in conjunction with or addition to the device. The device may be able to detect information related to voluntary user activities, such as without limitation, breathing, eating, swallowing, chewing, or drinking.

The user may after waking up consume water or other liquid. During the consumption of water, the device may receive a signal from an inner sensor, which may be analyzed by a first model. The first model may classify or determine that the signal corresponds to drinking. The first model may be run locally on the device and cause a second model to be implemented responsive to an output of the first model, such as a determination that “drinking” is taking place. The second model can be run on the device worn by the user or on a connected device, such as a laptop or smartphone. The results of the second model can be stored on provided to the connected device, such as in this example, the quantity of water consumed.

After hydrating, the user may wish to take a run or engage in another form of exercise. During exercise, the first model may determine that the user is only breathing, and cause a second model (e.g. a breathing model) to be initiated, which can determine information related to breathing. In this example, the second model may also take as inputs information from the connected health device, which may provide information related to SPO2, heart rate, or other heart or cardiovascular related information. The second model could analyze information from one or more sensors (e.g. inner microphone and thermometer) to analyze breathing rate, breathing depth, breathing patterns, and exertion by the user. The second model may also recognize that the user is likely engaging in a run based on gyroscope information received from the device or other worn wearable health device (e.g. smartwatch). The gyroscope information can also be used by the second model when analysing the breathing pattern, subtracting external signals from the sensors (e.g. wind noise), or interpreting other noise data (e.g. vibration or other noisy signals/data generated when the user's foot impacts a running surface). This information can be provided to a

Post-workout, the second model may continue to monitor the user's breathing during a cool down period. After a period of time, if a user decides to eat lunch or another meal, the first model could analyze that the user is engaging in chewing and swallowing, and cause a second model to analyze the cadence of chewing, swallowing, and estimate information related to lunch, such as the amount of food eaten or the estimated types of food eaten. At other times throughout the day, the first model can stand ready to analyze sensor data to detect when a user is eating, drinking, or engaging in other activities. The second model can analyze information related to each of these events.

At other times during the day, such as when a user is sneezing, coughing, or responding to other stimuli, a first model can analyze that a user is sneezing, coughing, congested, or otherwise responding to a stimulus, and a second model can be launched and can analyze sensor data, including the frequency of sneezing, coughing, breathing pattern, and/or temperature information to provide notifications to the user that the user may be sick, allergic to a particular object, congested, or be dehydrated.

As another example, the device may analyze a user's breathing patterns after detecting external sounds, such as a conversation, and analyze if there are any physiological responses to the sounds, such as elevated or decreased breathing rates, changes in breathing patterns, changes in body temperature, or other changes from a “baseline” behavior of the user.

In some examples, the use of two wearable devices or in-ear devices can allow for differential analysis between the signals received between the two devices to determine patterns of chewing, biting, bias in biting, grinding of teeth, or swallowing. For example, the signals received through a left ear and a right ear can be used differentially analyzed to make such determinations.

### Example Machine Learning, Statistical, Probabilistic, and Model Creation Methods

In some examples, one or more of the following techniques can be used as part of the disclosed technology.

In some examples, probabilistic methods can be used. For example, a gaussian mixture model can be used. Gaussian mixture models are a probabilistic model for representing normally distributed subpopulations within an overall population. In a Gaussian mixture model, it is not required that an observed set of data should characterize or state which subpopulation a particular observation within the distribution belongs to.

Example machine learning techniques which can be used include the following.

In some examples, a mix of supervised learning techniques and unsupervised learning techniques can be used.

In some examples, generative adversarial networks can be used to predict or detect network anomalies. Generative adversarial networks use two networks, one adversarial and one generative, in an attempt to fool the adversarial network by objects generated by the generative network.

In some examples, clustering methods can be used to cluster inputs, network parameters, trained models, or virtual machines. Clustering methods can be used in real time to classify and match models or groups of models with virtual machines or groups of virtual machines. Clustering can be an unsupervised machine learning technique in which the algorithm can define the output. One example clustering method is “K_Means” where K represents the number of clusters that the user can choose to create. Various techniques exist for choosing the value of K, such as for example, the elbow method.

Some other examples of techniques include dimensionality reduction. Dimensionality reduction can be used to remove the amount of information which is least impactful or statistically least significant. In networks, where a large amount of data is generated, and many types of data can be observed, dimensionality reduction can be used in conjunction with any of the techniques described herein. One example dimensionality reduction method is principle component analysis (PCA). PCA can be used to reduce the dimensions or number of variables of a “space” by finding new vectors which can maximize the linear variation of the data. PCA allows the amount of information lost to also be observed and for adjustments in the new vectors chosen to be made. Another example technique is t-Stochastic Neighbor Embedding (t-SNE).

Ensemble methods can be used, which primarily use the idea of combining several predictive models, which can be supervised ML or unsupervised ML to get higher quality predictions than each of the models could provide on their own. As one example, random forest algorithms

Neural networks and deep learning techniques can also be used for the techniques described above. Neural networks generally attempt to replicate the behavior of biological brains in turning connections between an input and output “on” or “off” in an attempt to maximize a chosen objective.

The technology can include any combination of the following features or aspects:
Aspect 1. An electronic device, comprising:
   one or more sensors;
   one or more processors; and
   one or more non-transitory computer-readable media that store instructions that when executed by the one or more processors cause the electronic device to perform operations, the operations comprising:
receiving sensor data generated by at least one sensor of the one or more sensors that is at least partially positioned within an ear of a user, wherein the sensor data was generated by the at least one sensor concurrently with a voluntary user activity;
processing the sensor data with a trained machine learning model to generate an interpretation of the voluntary user activity as an output of the trained machine learning model.
Aspect 2. The electronic device of aspect 1, wherein the at least one sensor comprises one or more microphones that convert a sound wave located within an ear canal of the ear of the user to the sensor data.
Aspect 3. The electronic device of aspect 2, wherein the sound wave located within the ear canal of the ear of the user is generated by an eardrum of the user.
Aspect 4. The electronic device of aspect 2 or 3, wherein, when the one or more microphones are placed within the ear of the user, the one or more microphones are directed toward the eardrum of the user.
Aspect 5. The electronic device of any one of the preceding aspects, wherein the at least one sensor comprises one or more of:
an accelerometer;
a gyroscope;
a RADAR device;
a SONAR device;
a LASER microphone;
an infrared sensor; or
a barometer.
Aspect 6. The electronic device of aspect 1, wherein the electronic device is sized and shaped to be at least partially positioned within the ear of the user.
Aspect 7. The electronic device of any of aspects 1-6, wherein the electronic device is coupled to an ancillary support device that is physically separate from the at least one sensor.
Aspect 8. The electronic device of any one of the preceding aspects, wherein the interpretation of the sensor data as an output by the trained machine learning model categorizes the sensor data as one or more of coughing, breathing, chewing, sneezing, swallowing, or drinking.
Aspect 9. The electronic device of any one of the preceding aspects, wherein the interpretation of the output by the trained machine learning model comprises a classification of the output into one or more of a plurality of categories.
Aspect 10. The electronic device of aspect 9, wherein the plurality of categories comprise a plurality of defined user states.
Aspect 11. The electronic device of any one of the preceding aspects, wherein the operations comprise:
determining, by analysis of additional health data obtained from a health sensor, whether an emergency condition related to the user is met.
Aspect 12. An ear bud, comprising:
at least one sensor;
one or more processors; and one or more non-transitory computer-readable media that store instructions that when executed by the one or more processors cause the ear bud to perform operations, the operations comprising:
receiving sensor data generated by the at least one sensor, wherein the sensor data was generated by the at least one sensor concurrently with user activity detectable through signals associated with a portion of the user's anatomy proximate the ear;
processing the sensor data with a machine-learned model to generate an interpretation of the sensor data as an output of the machine-learned interpretation model.
Aspect 13. The ear bud of aspect 12, wherein the at least one sensor comprises a microphone that converts a sound wave located within an ear canal of the ear of the user to the sensor data.
Aspect 14. The ear bud of aspect 13, wherein the sound wave located within the ear canal of the ear of the user is generated by an eardrum of the user.
Aspect 15. The ear bud of aspect 13 or 14, wherein, when the microphone is placed within the ear of the user, the microphone is directed to the eardrum of the user.
Aspect 16. The ear bud of any one of aspects 12 to 15, wherein the interpretation of the sensor data as an output by the trained machine learning model categorizes the sensor data as coughing, breathing, chewing, sneezing, swallowing, or drinking.
Aspect 17. The ear bud of any one of aspects 12 to 16, wherein the interpretation of the output by the machine-learned interpretation model comprises a classification of the output into one or more of a plurality of categories.
Aspect 18. The ear bud of aspect 17, wherein the plurality of categories comprise a plurality of defined user states.
Aspect 19. The ear bud of any one of aspects 12 to 18, wherein the operations comprise:
   determining, by analysis of additional health data obtained from a health sensor, whether an emergency condition related to the user is met.
Aspect 20. A method, comprising:
receiving sensor data generated by the at least one sensor, wherein the sensor data was generated by the at least one sensor concurrently with user activity detectable through signals associated with a portion of the user's anatomy proximate the ear; processing the sensor data with a machine-learned model to generate an interpretation of the sensor data as an output of the machine-learned interpretation model.
Aspect 21. The method of aspect 20, wherein the sensor data is based on sound waves produced by vibrations within the ear of the user resulting from the user's action.
Aspect 22. The method of aspect 20 or 21, wherein the sensor is part of an in-ear device.
Aspect 23. The method of aspect 22, wherein the in-ear device is coupled with at least one ancillary support device.
Aspect 24. The method of aspect 22 or 23, wherein trained machine learning model is implemented in the in-ear device.
Aspect 25. The method of aspect 23, wherein the trained machine learning model is implemented in the at least one ancillary support device.
Aspect 26. A system, comprising:
at least one sensor configured to be at least partially positioned within an ear of a user and to generate sensor data concurrently with user activity detectable through signals associated with a portion of the user's anatomy proximate the ear, one or more processors; and
one or more non-transitory computer-readable media that store instructions that when executed by the one or more processors cause the one or more processors to perform operations, the operations comprising:
   receiving the sensor data generated by the at least one sensor;
      processing the sensor data with a trained machine learning model to generate an interpretation of the user activity as an output of the trained machine learning model.
Aspect 27. The system of aspect 26, comprising processing health data obtained from a health sensor in conjunction with the sensor data.
Aspect 28. The system of aspect 26 or 27, wherein the sensor is part of an in-ear device of the system.
Aspect 29. The system of aspect 28, wherein the in-ear device is coupled with at least one user device of the system.
Aspect 30. The system of aspect 28 or 29, wherein the trained machine learning model is implemented in the in-ear device.
Aspect 31. The system of aspect 29, wherein the trained machine learning model is implemented in the at least one user device of the system.
Aspect 32. The system. Method, or device of any of the preceeding aspects wherein a first stage model and a second stage model can be used, the first stage model running on a first device, and a second stage model running on the second device, wherein the first stage model filters or categorizes the type of action, and the second stage model performs additional analysis related to the action, the first stage and second stage model being machine learning models.

While this disclosure contains many specific implementation details, these should not be construed as limitations on the scope of what may be claimed, but rather as descriptions of features specific to particular examples. Certain features that are described in this specification in the context of separate examples may also be implemented in combination in a single implementation. Conversely, various features that are described in the context of a single implementation may also be implemented in multiple examples separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

Various modifications to the examples described in this disclosure may be readily apparent to those skilled in the art, and the generic principles defined herein may be applied to other examples without departing from the spirit or scope of this disclosure. Thus, the claims are not intended to be limited to the examples shown herein, but are to be accorded the widest scope consistent with this disclosure, the principles and the novel features disclosed herein.

1. An electronic device, comprising:
one or more sensors;
one or more processors; and
one or more non-transitory computer-readable media that store instructions that when executed by the one or more processors cause the electronic device to perform operations, the operations comprising:
receiving sensor data generated by at least one sensor of the one or more sensors that is at least partially positioned within an ear of a user, wherein the sensor data was generated by the at least one sensor concurrently with a voluntary user activity;
processing the sensor data with a trained machine learning model to generate an interpretation of the voluntary user activity as an output of the trained machine learning model.

2. The electronic device of claim 1, wherein the at least one sensor comprises one or more microphones that convert a sound wave located within an ear canal of the ear of the user to the sensor data.

3. The electronic device of claim 2, wherein the sound wave located within the ear canal of the ear of the user is generated by an eardrum of the user.

4. The electronic device of claim 2, wherein, when the one or more microphones are placed within the ear of the user, the one or more microphones are directed toward the eardrum of the user.

5. The electronic device of claim 1, wherein the at least one sensor comprises one or more of:
an accelerometer;
a gyroscope;
a RADAR device;
a SONAR device;
a LASER microphone;
an infrared sensor; or
a barometer.

6. The electronic device of claim 1, wherein the electronic device is sized and shaped to be at least partially positioned within the ear of the user.

7. The electronic device of claim 1, wherein the electronic device is coupled to an ancillary support device that is physically separate from the at least one sensor.

8. The electronic device of claim 1, wherein the interpretation of the sensor data as an output by the trained machine learning model categorizes the sensor data as one or more of coughing, breathing, chewing, sneezing, swallowing, or drinking.

9. The electronic device of claim 1, wherein the interpretation of the output by the trained machine learning model comprises a classification of the output into one or more of a plurality of categories.

10. The electronic device of claim 9, wherein the plurality of categories comprise a plurality of defined user states.

11. The electronic device of claim 1, wherein the operations comprise:
determining, by analysis of additional health data obtained from a health sensor, whether an emergency condition related to the user is met.

12. A method, comprising:
receiving sensor data generated by at least one sensor of the one or more sensors that is at least partially positioned within an ear of a user, wherein the sensor data was generated by the at least one sensor concurrently with a voluntary user activity;
processing the sensor data with a trained machine learning model to generate an interpretation of the voluntary user activity as an output of the trained machine learning model.

13. The method of claim 12, wherein the sensor data is based on sound waves produced by vibrations within the ear of the user resulting from a user activity or action.

14. The method of claim 12, wherein the sensor is part of an in-ear device.

15. The method of claim 14, wherein the in-ear device is coupled with at least one ancillary support device.

16. The method of claim 12, wherein trained machine learning model is implemented in the in-ear device.

17. The method of claim 16, wherein the trained machine learning model is implemented in an ancillary support device.

18. A system, comprising:
at least one sensor configured to be at least partially positioned within an ear of a user and to generate sensor data concurrently with user activity detectable through signals associated with a portion of the user's anatomy proximate the ear,
one or more processors; and
one or more non-transitory computer-readable media that store instructions that when executed by the one or more processors cause the one or more processors to perform operations, the operations comprising:
receiving the sensor data generated by the at least one sensor;
processing the sensor data with a trained machine learning model to generate an interpretation of the user activity as an output of the trained machine learning model.

19. The system of claim 18, comprising processing health data obtained from a health sensor in conjunction with the sensor data.

20. The system of claim 18, wherein the sensor is part of an in-ear device of the system.
